(19) United States
(12) Patent Application Publication
(10) Pub. No.: US 2005/0192645 A1
(43) Pub. Date: Sep. 1, 2005

(54) Method to produce a balanced dorsiflexion during the gait of patients with foot drop

(76) Inventors: Richard B. Stein, Edmonton (CA); K. Ming Chan, Edmonton (CA); Douglas J. Weber, Edmonton (CA); Robert Rolf, Edmonton (CA)

Correspondence Address: SHERIDAN ROSS PC, 1560 BROADWAY, SUITE 1200, DENVER, CO, 80202, US

(21) Appl. No.: 11/046,529

(22) Filed: Jan. 28, 2005

Related U.S. Application Data
(60) Provisional application No. 60/540,234, filed on Jan. 28, 2004.

(51) Int. Cl. A61N 1/18

(52) U.S. Cl. 607/49

(57) ABSTRACT

A ministimulator is positioned adjacent to the deep peroneal nerve and electrically actuated to elicit balanced dorsiflexion, without eversion, of the ankle of a patient having foot drop.

### REFERENCE TO RELATED APPLICATION

The present application is related to U.S. Provisional Patent Application Ser. No. 60/540,234, entitled “A Method to Produce a Balanced Dorsiflexion During the Gait of Patients with Foot Drop”, filed Jan. 28, 2004, which is incorporated herein by reference in its entirety.

### FIELD OF THE INVENTION

The invention relates to implanting a miniature electrical stimulator (“microstimulator”) in the leg of a patient and applying stimulation for the purpose of alleviating foot drop.

### BACKGROUND OF THE INVENTION

Foot drop is a common consequence of diseases affecting peripheral nerves or areas of the central nervous system that normally produce dorsiflexion of the ankle during the swing phase of walking. If the appropriate nerves are not activated, the foot drops and may drag on the ground instead of swinging smoothly through the air. If the cause of the problem is central in origin the peripheral nerves are still available for stimulation.

Liberson et al¹ in 1961 first proposed that the stimulation of the common peroneal (“CP”) nerve could be timed appropriately using a heel switch to turn on when the heel leaves the ground and turn off when the heel again hits the ground. More recently, Stein, in U.S. Pat. No. 5,814,093, incorporated herein by reference, taught that a tilt sensor built into a foot drop stimulator could improve CP nerve stimulation using external electrodes by relating leg position during gait with the initiation and termination of stimulation. The patent taught an electronic circuit for so controlling stimulation in response to tilt sensor readings.

However, externally applied stimulation of the CP nerve innervates muscles that flex the ankle (e.g. the tibialis anterior, “TA,” muscle and the extensor digitorum longus, “EDL”, muscle) and others (e.g. peroneus longus, “PL”, muscle) that evert the ankle (i.e. rotate it outward). Furthermore, the PL muscle is innervated by the superficial branch of the CP nerve and so is more easily stimulated from skin surface. Obtaining a balanced dorsiflexion (that is, without significant eversion) of the ankle with surface electrodes is therefore difficult.

There have been attempts described in the literature for resolving this problem by surgically implanting electrodes near the CP nerve or near the motor points of more than one muscle (O'Halloron et al², 2003; Rozman et al³, 1990; Waters et al⁴, 1973). The former approach does not solve the problem since stimulating the whole nerve via surface electrodes or subcutaneously will still produce eversion as well as flexion. The latter approach is problematic since the motor points of these muscles are often quite distributed in space and several muscles and motor points may need to be stimulated.

In recent years, small injectable microstimulators were developed by Schulman et al and disclosed in U.S. Pat. Nos. 5,324,316 and 5,405,367 (both incorporated herein by reference). These microstimulators can be inserted into tissue using a hypodermic needle, without surgery.

### SUMMARY OF THE INVENTION

This invention is based on the discovery that if a microstimulator is implanted in a patient's leg at a location or implantation site adjacent to the deep peroneal (“DP”) nerve and remote from the common peroneal (“CP”) nerve and its superficial branch (known as the superficial peroneal nerve or “SP” nerve), and if the microstimulator is energized to stimulate the DP nerve during the swing phase of gait, then such stimulation will elicit balanced dorsiflexion of the patient's ankle, substantially without eversion.

In order to take advantage of this discovery, it was necessary to develop:
a workable system for positioning and delivering the microstimulator to the aforementioned site, where it would preferably lie generally parallel and adjacent to and at substantially the same depth as the DP nerve, at a locus beneath the PL muscle and spaced forwardly of the anterior tibial artery; and
a hardware system for controlling and implementing stimulation of the DP nerve in relation to gait.

This involved: mapping, in connection with the patient's leg, the path or course of the CP nerve, the branch point for the SP and DP nerves, and the course of the latter nerve; selecting an implantation site along the course of the DP nerve; determining the depth of the DP nerve at the site; and implanting the microstimulator at the site so as to lie adjacent to and preferably alongside the DP nerve.

It further involved: monitoring the position of the patient leg during gait; and initiating and terminating electrical stimulation of the DP nerve alone during the swing phase of gait so as to elicit balanced dorsiflexion of the ankle.

In one embodiment, the invention is concerned with a method for treating a patient with foot drop, comprising: implanting a microstimulator, adjacent to the DP nerve of the patient's leg, for delivering stimulus thereto; and electrically stimulating the DP nerve to elicit balanced dorsiflexion of the patient's ankle.

In another embodiment, the invention is concerned with a method for treating a patient with foot drop, comprising: mapping, in connection with the patient's leg, the course of the CP nerve, the branch point for the SP nerve and the DP nerve from the CP nerve, and the course of the DP nerve; selecting an implantation site adjacent to the course of the DP nerve; determining the depth of the DP nerve at the site; implanting a microstimulator at the site adjacent the DP nerve; and electrically stimulating the DP nerve to elicit balanced dorsiflexion of the patient's ankle.

### DESCRIPTION OF THE DRAWINGS

FIG. 1 is a side view of part of the anatomy of a human leg, showing the proposed insertion path and implantation site for a ministimulator, in accordance with the invention;

FIG. 2 is a photograph showing a patient leg in the process of mapping the courses of the CP nerve, the SP nerve and the DP nerve;

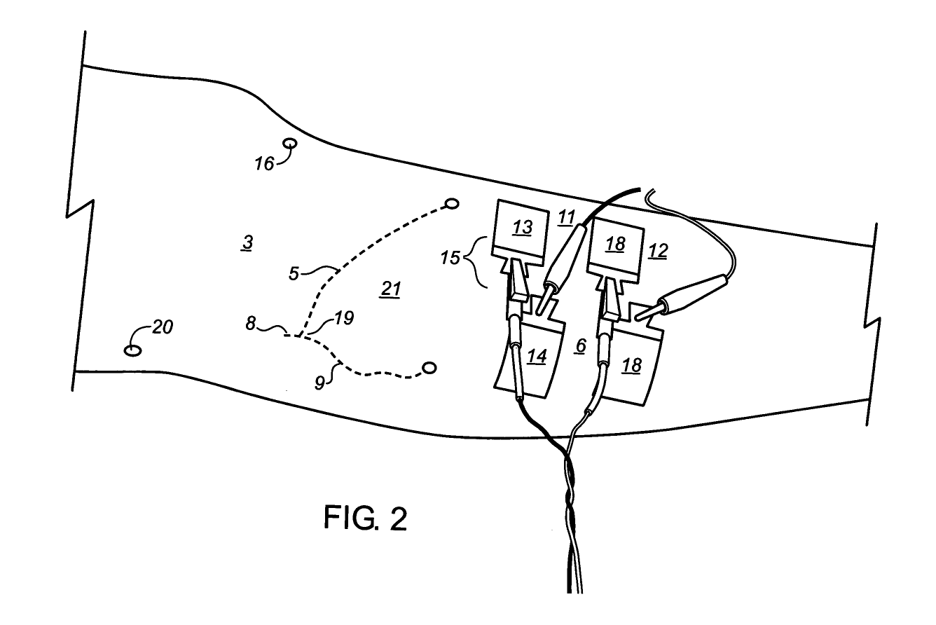

FIG. 3 is a photograph showing the leg in the process of determining the depth of the implantation site;

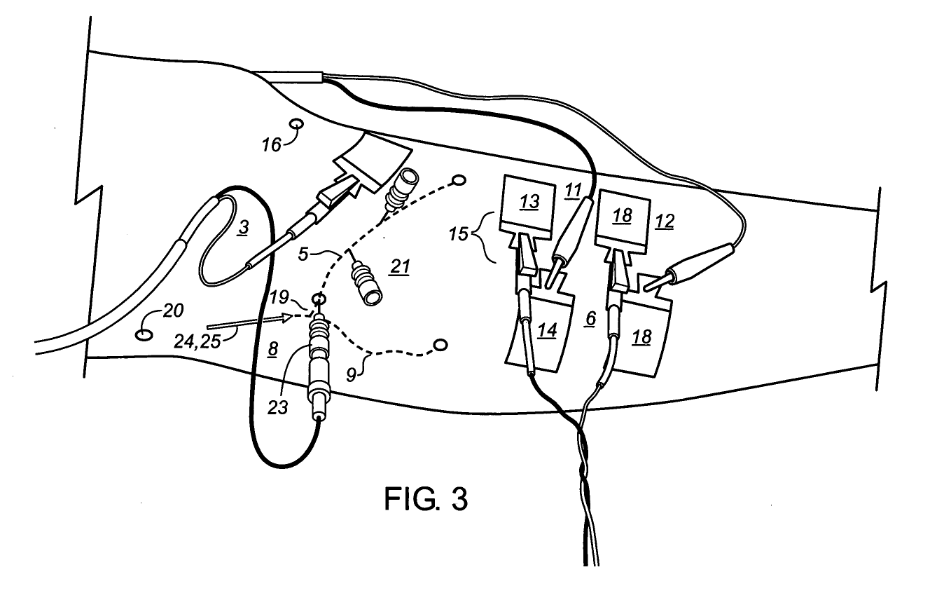

FIG. 4 is a schematic block diagram of the system used to monitor leg gait and control nerve stimulation in response thereto; and

FIG. 5 is a more detailed schematic block diagram of the system shown in FIG. 4.

### DESCRIPTION OF THE PREFERRED EMBODIMENT

This description teaches:
a technique for accurately positioning and implanting a microstimulator 1 at a desired site 2 in a patient's leg 3; and
a system 4 for energizing and controlling the timing and duration of stimulation.

More particularly, a suitable implantable microstimulator 1 is the Schulman et al device previously mentioned and available from the Alfred E. Mann Foundation for Scientific Research, Sylmar, Calif. and the Alfred E. Mann Institute at the University of Southern California, Los Angeles, Calif. This microstimulator 1 and its associated equipment is identified by the trade-mark BION. The microstimulator 1 is energized and controlled using radio frequency signals from a custom circuit, forming part of the supplied BION equipment. This microstimulator 1 can be implanted through a hypodermic needle, such as an Angiocath™ needle. This device comprises of a plastic sheath surrounding a hypodermic needle. Once the needle is withdrawn, when its tip has reached the desired site a Bion microstimulator can be pushed down the plastic sheath by using a plunger, to sit at the location formerly occupied by the needle tip.

The microstimulator 1 is to be positioned in the patient's leg, substantially parallel and adjacent to the DP nerve 5 at an implantation site 2 immediately beneath the PL muscle 6 and spaced forwardly of the anterior tibial artery 7. The selected site 2 should be sufficiently remote from the CP and SP nerves 8,9 and sufficiently close to the DP nerve 5 so that low intensity stimulation (e.g. 1-3 microamps) by the microstimulator 1 will activate the TA and EDL muscles 11, 12 while the PL muscle 6 remains quiescent.

This can be achieved in the following manner.

By way of overview, electromyographic (“EMG”) recordings from several muscles are used to map the courses of the CP, SP and DP nerves 8, 9, 5. These recordings are developed in the following manner.

Two sets 13, 14 of surface self-adhesive EMG recording electrodes 15 are placed on the skin of the patient's leg 3. One set 13 is placed over the belly of the TA muscle 11. This set 13 also records to some extent from the nearby EDL muscle 12. The other set 14 is placed over the PL muscle 6. The recording electrode sets 13, 14 are placed directly over the relevant motor point, which is usually located 4 finger breadths distal to the tibial tuberosity 16 in the case of the TA muscle 11 and 7 finger breadth below the fibular head 17 in the case of the PL muscle 6.

After the EMG electrode sets 13, 14 and associated conventional EMG equipment are so positioned and operatively connected, a bipolar hand-held stimulator is used to surface stimulate the CP nerve 8 and its branches—the DP and SP nerves 5, 9. Further refinement of the precise location of the site 2 can be achieved by moving the recording electrode until a location where the amplitude of the maximum motor (“M”) wave produced by stimulating the nerves is greatest and the rising slope of the wave is sharpest. A reference electrode 18 is placed 5 cm distally to each of the relevant recording electrodes. This bipolar configuration helps to minimize noise in the recording and improves selectivity of the recorded target muscles. The goal is to record from muscles innervated by the DP and SP nerves as selectively as possible.

The courses of the CP, SP and DP nerves 8, 9, 5 and the location of the branch point 19, between the popliteal fossa 20 and the proximal calf 21, are mapped out by moving the stimulating electrode and finding the locations at which the largest M-wave can be elicited using the lowest stimulus intensity. Activation of the nerve can only be achieved by low stimulus currents when the stimulating electrode is in close proximity to the nerve. Further confirmation of activating of the target nerves can also be obtained by observing the mechanical twitch of the innervated muscles. As long as the stimulus is over the CP nerve 8 large M-waves will be recorded from both sets 13, 14 of EMG electrodes. Once the branch point is passed, and the stimulating electrode is over the DP nerve, only a large TA muscle M-wave will be recorded. Conversely, if the stimulating electrode is over the SP nerve only a large PL muscle M-wave will be recorded.

Once the courses of each of the nerves 8, 9, 5 and the branch point 19 have been mapped, the depth of the DP nerve 5 under the skin can be established using a fine monopolar needle electrode 23.

The needle electrode 23 is inserted close to the DP nerve 5, about 2 cm beyond the branch point 19. Once the needle electrode 23 has been inserted perpendicularly to the skin, the stimulus intensity is gradually increased until a clear, reproducible M-wave can be elicited. This intensity is a measure of the distance of the electrode 23 from the nerve and will decrease as the distance decreases.

The needle electrode 23 is then carefully advanced perpendicularly into the leg tissue in small increments. At each new depth, stimulation is repeated and the stimulus intensity needed to produce an M-wave of the same amplitude is noted. Further advancement of the needle electrode 23 is halted when a point at which very low stimulus intensity requirement (in the region of 1 to 3 mA with a rectangular pulse width of 200 microseconds) is reached. A nerve can only be activated at such a low intensity if the electrode is very close to the nerve.

This first needle electrode 23 is then left in place. The depth of the needle tip can be estimated by measuring the length of the remaining part of the needle electrode protruding above the skin.

Thus, the target implantation site 2 to which to direct the microstimulator is known in three dimensions, two along the skin surface and the third in terms of the depth of the nerve below the skin.

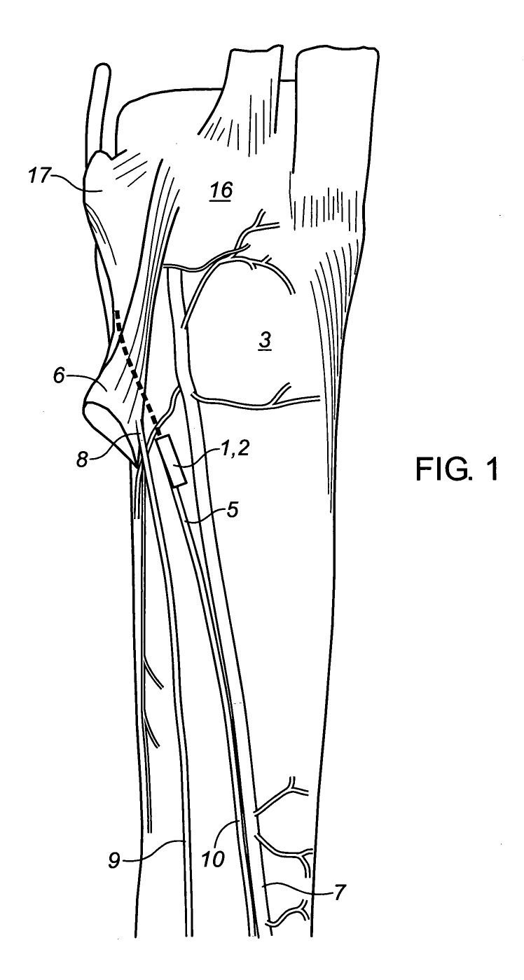

Insertion of the implantation tool, a hypodermic needle 25, is now initiated. The hypodermic needle 25 is a modified 12 gauge Angiocath™ needle that allows electrical stimulation through the trocar tip. The hypodermic needle 25 is inserted along the path 24 shown in FIG. 1. This path 24 follows the CP nerve 8 past the branch point 19 and then along the DP nerve 5. At each step, single stimulation pulses are applied

As the hypodermic needle 25 is advanced along the insertion path 24, it initially excites both TA and PL muscles 11, 6, since it is following the path of the CP nerve 8. However, one can feel a difference in resistance to insertion when the needle 25 reaches the tendinous origin of the PL muscle 6. Once the needle 25 goes through the PL muscle 6, it again moves more easily and the TA muscle 11 is stimulated selectively at levels similar to that obtained by the original needle electrode 23. Then, the two needles 23, 25 are close to each other and to the DP nerve 5. The tip of the hypodermic needle 25 is now at the desired site 2 and the needle electrode 23 can be removed.

When the tip of the hypodermic needle 25 has been placed at the desired microstimulator implantation site 2, the trocar is removed. A microstimulator is inserted into the lumen of the needle 25. A plunger is then used to apply a light pushing force to the back end of the microstimulator to eject it into the leg tissue.

The hypodermic needle 25 is then removed and the microstimulator is tested for functionality and the motor threshold is measured. Testing is done by placing the microstimulator coil 26 over the implant site 2. Stimulation pulses are applied in increasing steps until a noticeable muscle twitch in TA muscle 11 is produced. Increasing the stimulation intensity should produce a brisk muscle twitch and a large TA muscle M-wave with little or no PL muscle M-wave. This indicates that the microstimulator is in the desired position. Then, the stimulation is discontinued for 4-6 days to allow the surrounding tissue to heal. If the microstimulator is not properly positioned to give selective stimulation of the TA muscle, the process can be repeated with a second microstimulator A similar threshold test is performed to create a history of thresholds for each microstimulator, if more than one have been implanted.

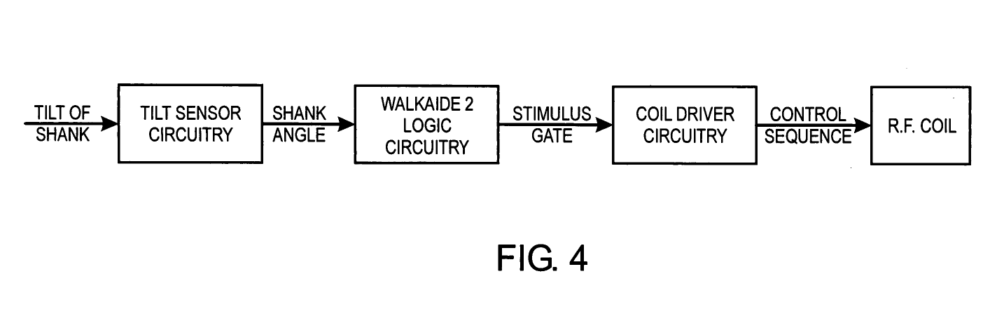

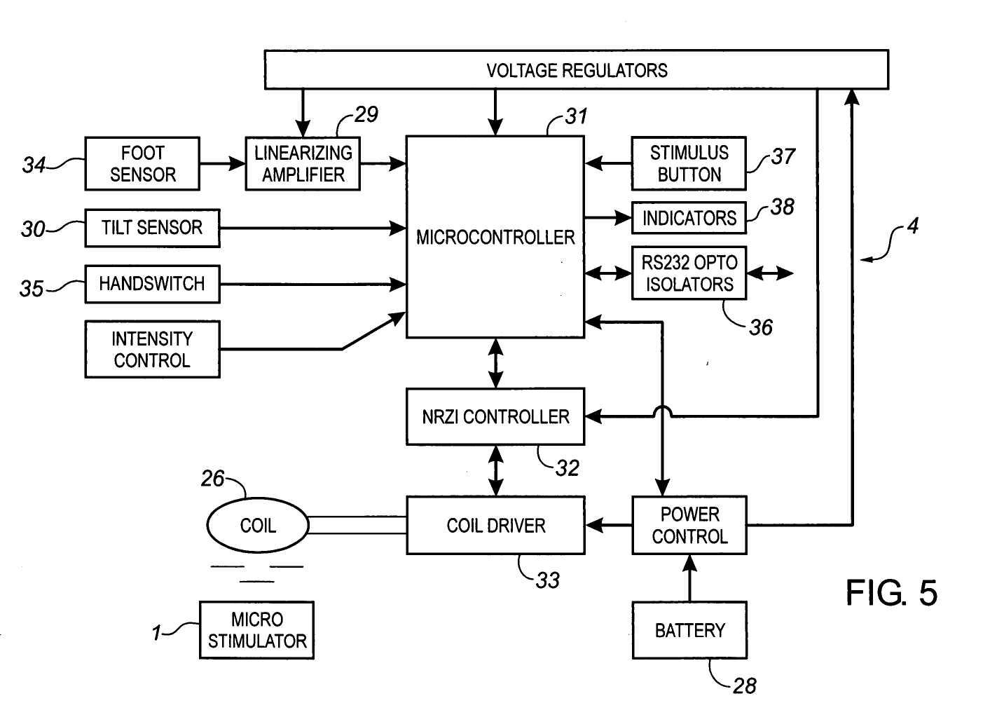

Having reference now to FIGS. 4 and 5, there are shown general and more specific schematic block diagrams of the system for driving implanted microstimulators and controlling the timing and duration of stimulations. This system combines the BION™ hardware and the Walk Aide 2™ hardware available from Biomotion Ltd., Edmonton, Alberta and described in U.S. Pat. No. 5,814,093.

In connection with the Walk Aide 2 hardware, a tilt of the leg shank backwards relative to the body at the end of the stance phase of the walking cycle activates tilt sensor circuitry 30 that sends a signal representing tilt angle to microcontroller 31. If the tilt signal exceeds a predetermined threshold and some other logic conditions are met, for example that stimuli have not been generated for a period known as the “Wait” period, a stimulus gate signal is generated. This signal is formatted as a code sequence that can be decoded by the Bion microstimulator 1 to produce a pattern of stimuli with the desired amplitude and duration. In the preferred implementation the sequence of commands is formatted efficiently using a non-return to zero invert (NRZI) formatter 32. The coded sequence is then sent to the BION coil driver circuit 33 and then to the coil 26. The microstimulator internal circuitry decodes this sequence and produces a prescribed sequence of stimulus pulses. The BION microstimulator 1 contains no batteries, so the external coil 26 must supply power as well as the sequence of control pulses. The block diagram of FIG. 5 also shows other sensors and controls that enhance the flexibility of the overall design.

In greater detail, a lithium ion battery (7.2V) 28 is used to power both the coil driver 33 and the other electronics (after regulation to 5V). The coil driver 33 is tuned to the preferred radio frequency of the microstimulator 1 and is shaped to fit in a cuff around the leg, so that it covers the implanted microstimulator(s) 1.

A combination of sensors is used to control the timing of the stimulation. More particularly, a tilt sensor 30 (Analog Devices ADXL202) measures the orientation of the leg with respect to gravity, a foot sensor 34 (Interlink Technology force sensing resistor FSR-20) measures the pressure of the heel on the ground and a hand switch 35 can be used by a clinician to set up the initial timing of the stimuli.

A linearizing amplifier 29 corrects the otherwise non-linear response of the foot sensor 34.

The output of the tilt sensor 30 is filtered (not shown) to remove sharp transients such as the deceleration of the foot hitting the ground.

The microcontroller 31 (Microchip PIC16LF 876) processes inputs and generates outputs based on a state engine that includes timing constraints, as described in U.S. Pat. No. 5,814,093. The microcontroller 31 has on-board non-volatile memory for storage of parameters used by the state machine. The parameters can be adjusted using a Windows™ program, Walk Analyst™, which allows the stimulation current, the duration and the frequency of the stimuli produced by the microstimulator(s) 1 to be varied as desired for optimum function. The parameters are read and written via serial communications with the microcontroller 31 using the optically isolated RS232 interface isolator 36.

The stimulus button 37 allows the operation of the electronics and the positioning of the coil 26 to be tested, as well as allowing the users to adjust the intensity control to the desired level.

Indicators 38 are provided for off/on status, stimulation and low battery conditions.

In previous implementations for surface stimulation the microcontroller produced pulses that were amplified to produce stimuli directly to the muscles through the skin. In the current implementation, a string of non-return to zero invert (NRZI) encoded data is generated that modulates the frequency of the coil 26 and conveys the stimulus parameter to the microstimulator 1. The NRZI formatter 32 offloads the overhead of encoding and maintaining an ‘idle’ (recharge) power condition in the microstimulator 1 by using a recirculating shift register. The formatter circuit therefore reduces the speed requirements for the microcontroller in communicating with the microstimulator, by taking care of synchronization and data encoding issues that would usually be done with firmware. This results in power savings by allowing a lower system clock speed that would otherwise be needed to supply the coil with data for the microstimulator.

Although particular devices used have been identified in the foregoing description, the invention is not limited to these devices. Other implanted stimulation devices that are sufficiently small to fit in the space available should also work. Also, other foot drop stimulators could be modified to drive the microstimulator appropriately.

### REFERENCES

(1) Liberson, W. T., Holmquest, H. J., Scott, D., and Dow, M. 1961. Functional electrotherapy, stimulation of the peroneal nerve synchronized with the swing phase of the gait of hemiplegic patients. Arch. Phys. Med., 42: 101-105;
(2) O'Halloran, T., Haugland, M., Lyons, G. M., and Sinkjaer, T. 3004. Modified implanted drop foot stimulator system with graphical user interface for customized stimulation pulse-width profiles. Med. Biol. Eng. Comput, 41(6): 701-709;
(3) Rozman, J., Stanic, U., Malezic, M., Acimovic-Janezic, R., Kljajic, M., and Kralj, A. 1990. Implantable electrical stimulation and technology of Jozef Stefan Institute in Ljubljana. In Advances in External Control of Human Extremities. Nauka, Belgrade, Yugoslavia. pp. 617-626.
(4) Waters, R. L., McNeal, D., and Perry, J. 1975. Experimental correction of foot drop by electrical stimulation of the peroneal nerve. Journal of Bone and Joint Surgery, 57A: 1047-1054.

1. A method for treating a patient with foot drop, comprising:
implanting a microstimulator, adjacent to the deep peroneal (“DP”) nerve of the patient's leg, for delivering stimulus thereto; and
electrically stimulating the DP nerve to elicit balanced dorsiflexion of the patient's ankle.

2. The method as set forth in claim 1 wherein the microstimulator is implanted generally parallel with and substantially at the same depth as the DP nerve.

3. The method as set forth in claim 1 wherein the DP nerve is electrically stimulated to elicit balanced dorsiflexion without substantial eversion of the patient's ankle.

4. The method as set forth in claim 2 wherein the DP nerve is electrically stimulated to elicit balanced dorsiflexion without substantial eversion of the patient's ankle.

5. A method for treating a patient with foot drop, comprising:
mapping, in connection with the patient's leg, the course of the common peroneal (“CP”) nerve, the branch point for the superficial peroneal (“SP”) nerve and the deep peroneal (“DP”) nerve from the CP nerve, and the course of the DP nerve;
selecting an implantation site adjacent the course of the DP nerve;
determining the depth of the DP nerve at the site;
implanting a microstimulator at the site adjacent the DP nerve; and
electrically stimulating the DP nerve to elicit balanced dorsiflexion of the patient's ankle.

6. The method as set forth in claim 5 wherein the microstimulator is implanted generally parallel with and substantially at the same depth as the DP nerve.

7. The method as set forth in claim 5 wherein the DP nerve is electrically stimulated to elicit balanced dorsiflexion without substantial eversion of the patient's ankle.

8. The method as set forth in claim 6 wherein the DP nerve is electrically stimulated to elicit balanced dorsiflexion without substantial eversion of the patient's ankle.
